(19) United States
(12) Patent Application Publication
(10) Pub. No.: US 2005/0032900 A1
(43) Pub. Date: Feb. 10, 2005

(54) Ibuprofen salt emulsifiers and cream formulations containing same

(76) Inventor: Scott F. Krauser, Westford, MA (US)

Correspondence Address: MAYER, BROWN, ROWE & MAW LLP, 1909 K STREET, N.W., WASHINGTON, DC, 20006, US

(21) Appl. No.: 10/491,493

(22) PCT Filed: Oct. 4, 2002
(86) PCT No.: PCT/US02/31798

Related U.S. Application Data
(60) Provisional application No. 60/326,718, filed on Oct. 4, 2001.

(51) Int. Cl. A61K 31/192; A61K 9/00

(52) U.S. Cl. 514/570; 424/400

(57) ABSTRACT

Emulsions and creams containing ibuprofen effective for topical administration of the ibuprofen are provided without the presence of conventional O/W emulsifying agent. The emulsions and creams are formed by using the ibuprofen, in the form of its salt, as the O/W emulsifying agent. A minor amount of a W/O emulsifying agent may be included in the composition. Percutaneous delivery of ibuprofen is improved by using a skin penetration enhancing compound, such as 2-n-nonyl-1,3-dioxolane or decanaldimethyl acetal, as the oily phase of the emulsion.

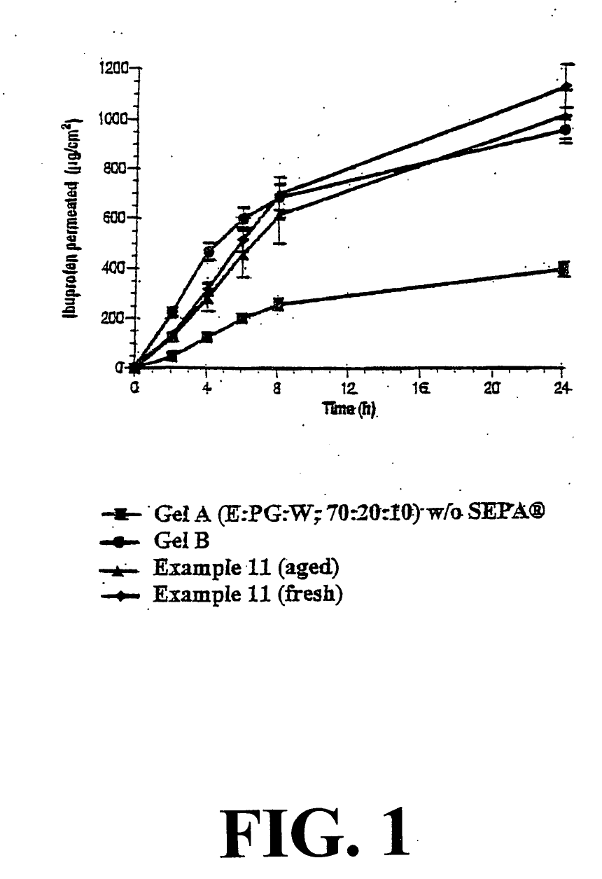

This application claims priority to U.S. Provisional Application Ser. No. 60/326,718, filed Oct. 4, 2001.

### FIELD OF INVENTION

This invention relates to the discovery that ibuprofen salts are effective as emulsifiers and can be used to formulate cream compositions which are stable and effective as delivery vehicles for delivering therapeutically effective doses of ibuprofen to the slkin. More particularly, this invention relates to substantially neutral cream formulations containing ibuprofen, in the form of its salt, as emulsifier and as active ingredient.

### STATEMENT OF PRIOR ART

Compositions designed for the topical administration of ibuprofen (α-methyl-4-(2-methylpropyl)benzene acetic acid; or 2-(4-isobutylphenyl)-propionic acid) are known, and in some parts of the world, have been commercially available. For example, the following patent literature is considered to be representative of disclosures which may be considered relevant to the present invention: U.S. Pat. No. 4,514,386 to Y. Yamahira, et al; U.S. Pat. No. 4,555,524 to K. Gruber, et al; U.S. Pat. No. 5,093,133 to Wisniewslki, et al; U.S. Pat. No. 5,104,656 to P Seth, et al; U.S. Pat. No. 5,210,099 to D Mody; U.S. Pat. No. 5,318,960 to F. Toppo; U.S. Pat. No. 5,510,302 to G Atkin, et al; U.S. Pat. No. 5,527,832 to S-C. Chi, et al; U.S. Pat. No. 5,654,337 to E. Roentsch; U.S. Pat. No. 5,985,860 to F. Toppo; U.S. Pat. No. 6,211,250 to R. Tomlinson, et al; GB 2236250 to Kenmeth M. Henderson; WO 91/04733 to The Mentholatum Company; WO 98/25995 to The Boots Company; WO 01/02015 to J. H. Won, et al.

A self-emulsifying ibuprofen solution for use in soft gelatin capsules is the subject of U.S. Pat. No. 6,221,391 to Rouffer.

Notwithstanding the substantial interest in developing safe and effective topical delivery systems for the percutaneous (through the skin) delivery of analgesic and anti-inflammatory drugs, including ibuprofen, and the commonality of incorporating active agents in gels, creams, lotions, ointments, and the like, it was never reported in the literature, as far as the present applicant is aware, that ibuprofen, itself, is effective as an oil-in-water emulsifying agent, under substantially neutral conditions (e.g., between about pH 5 to about pH 9). In fact, it is generally considered to be difficult to formulate useful ibuprofen emulsions, insofar as addition of ibuprofen to many standard emulsion systems tends to break the emulsion.

Similarly, based on extensive work of the present inventor to formulate useful emulsions containing liquid type skin penetration enhancing compounds, especially 2-n-nonyl-1,3-dioxolane (commercially available, under the trade name SEPA®-0009, from MacroChem Corp, Lexington, Mass.), it was also known that addition of such additives tends to destabilize the emulsion.

Accordingly, it was quite surprising when the present inventor discovered, in the course of preparing a conventional ibuprofen cream formulation, in combination with an oily skin penetration enhancer, that even before the addition of a conventional emulsifying agent, the combination of ibuprofen, 2-n-nonyl-1,3-dioxolane and water, in the presence of a small amount of base, formed a homogenous composition, wherein the partially neutralized ibuprofen salt functioned as an emulsifying agent.

The present invention is based on this discovery by the applicant.

### SUMMARY OF THE INVENTION

The present invention provides ibuprofen o/w emulsions comprising:
an emulsifying and therapeutically effective amount of ibuprofen salt;
oily substance, and,
water.

In a preferred embodiment of the invention, the oily substance is a skin penetration enhancing compound.

In still another aspect of the invention, the ibuprofen emulsion is converted into a cream formulation by addition of a thickening agent.

The compositions (emulsions and creams) may further include a minor amount, relative to the emulsifying amount of ibuprofen, of a secondary W/O emulsifying agent.

The compositions of this invention may also include other additives commonly included in topical emulsion and cream formulations, such as, for example, preservatives, odorants or perfumes, and the like.

In another aspect, the present invention provides a method for forming an ibuprofen cream formulation, wherein ibuprofen (in free acid form) is added to a mixture of aqueous solution of a base and an oily substance, to form an at least substantially homogeneous emulsion, and thereafter, adding a thickening agent to the emulsion to form a cream.

In one particular embodiment, the mixture of the aqueous solution of a base and an oily substance contains a minor amount of a secondary W/O emulsifying agent.

In another, related embodiment, a minor amount of secondary W/O surface active agent is added to the homogeneous emulsion before the addition of the thickening agent.

The present invention also provides a method for the transdermal delivery of ibuprofen from a cream formulation using the ibuprofen-containing cream as described above.

### BRIEF DESCRIPTION OF THE DRAWING

The attached Figure is a group plotting permeation (μg/cm²) of ibuprofen in a standard diffusion cell, as a function of time (h) for Gel A ( [symbol] ), Gel B ( [symbol] ), Example 11 ( [symbol] ) (fresh) and Example 11 ( [symbol] ) (aged).

### DETAILED DESCRIPTION AND PREFERRED EMBODIMENTS

According to the present invention O/W emulsions containing ibuprofen are obtained without the use of conventional O/W emulsifying compounds. This is made possible by the use of salts of ibuprofen as emulsifying agent.

The base, which may be used in the present invention, is not particularly critical and may be any water soluble inorganic or organic basic material which is safe for contact with human skin. As examples of an inorganic base, mention may be made of water soluble alkali metal and alkaline earth metal salts, such as, sodium hydroxide, potassium hydroxide, sodium carbonate, potassium carbonate, and mixtures thereof. As examples of organic basic materials, mention may be made of amines, such as, for example, alkyl amines, dialkyl amines and trialkyl amines, preferably wherein the alkyl group has from 1 to 6 carbon atoms, which, in the case of the dialkyl amines and trialkyl amines may be the same or different, e.g., ethyl amine, isopropylamine, methylethylamine, butyl amine, diethylamine, diisopropylamine, triethylamine, and the like; dialkyl and polyamines, such as, ethylenediamine, alkanolamines, such as, for example, diethanolamine, triethanolamine, diisopropanolamine, and the like.

The base may be added in an amount to neutralize a portion of the carboxylic groups of ibuprofen, generally, up to about 0.8 mole of base, per mole of ibuprofen, preferably, from about 0.1 to about 0.7 mole of base per mole of ibuprofen, especially from about 0.2 to about 0.6 mole of base per mole of ibuprofen. Usually, the amount of base will provide a substantially neutral to slightly acidic or basic pH. Particularly, depending on the sensitivity of any other ingredient to pH, the amount of base may be appropriately determined, however, usually, the compositions will be provided with sufficient base to provide a pH in the range of from, about 4 to about 8, preferably,. from about 4.3 to about 7.8, especially preferably, from about 6.0 to about 7.5.

The amount of ibuprofen will preferably be selected to provide not only the necessary degree of emulsification but also a therapeutically effective amount of ibuprofen as non-steroidal anti-inflammatory agent (NSAID), e.g., for its analgesic and/or anti-inflammatory effect. Accordingly, those skilled in the art will be able to determine a suitable amount of ibuprofen, depending on the intended use of the resulting emulsion or cream. Generally, however, amounts within the range of from about 1 to 10% by weight, such as from about 2 to 8%, preferably from about 3% to about 8%, such as about 5%, of ibuprofen, based on the weight of the emulsion or. cream, will provide an emulsifying and therapeutically effective level of ibuprofen effective for topical application to the skin of a human or other mammal.

The oil phase of the emulsion may be formed from any oily substance, such as, those used in the cosmetic or pharmaceutical field for preparation of emulsions. For example, mention may be made of mineral oil, silicone oil, e.g., cyclomethicone, triglycerides, e.g., C₆₋₁₂ carboxylic acid triglycerides, such as caprylic/capric triglyceride, and the like.

The amount of the oil phase is not particularly critical, consistent with the formation of the desired O/W emulsion, however, usually, amounts of the oil phase up to about 20%, preferably, up to about 15% of the emulsion, will be readily emulsified by the ibuprofen salt. Accordingly, amounts of oily phase in the range of from about 1 to about 20% by weight of the emulsion, preferably, from about 2 to about 15%, by weight, will form satisfactory emulsion compositions.

In a particularly preferred embodiment of the invention, the oil phase is comprised of a skin penetration enhancing compound having at least one fatty alkyl group substituent with 6 or more carbon atoms, preferably, 7 or more carbon atoms, such as from about 8 to about 20 carbon atoms. Non-limiting examples of skin penetration enhancing (SPE) compounds which may advantageously be used in the subject emulsion and cream formulations, include, one or more compounds from the following classes,
(i) C₇ to C₁₄-hydrocarbyl substituted 1,3-dioxolane, 1,3-dioxane or acetal;
(ii) macrocyclic ketones and lactones and derivatives thereof;
(iii) alkyl-2-(N,N-disubstituted amino)-alkanoate ester, (N,N-disubstituted amino)-alkanol alkanoate, or mixture thereof;
(iv) N-alkyl lactams and N-alkyl azacycloheptanes;
(v) fatty acid esters.
   In addition, mixtures of two or more enhancer compounds from any or a mixture of these groups may also be used.

The SPE (i) includes the substituted 1,3-dioxacyclopentane and substituted 1,3-dioxacyclohexane types disclosed in U.S. Pat. No. 4,861,764, the disclosure of which is incorporated herein in its entirety by reference thereto, or the corresponding substituted acetal compound. Representative examples of the skin penetration enhancing compounds include:
2-substituted 1,3-dioxolanes of the formula (I): 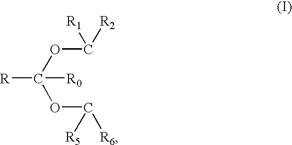
2-substituted 1,3-dioxanes of the formula (II): 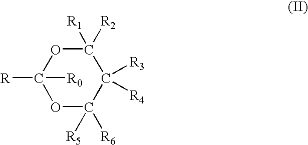
substituted-acetals of the formula (III):

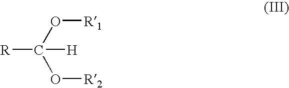

In the above formulas (I), (II) and (III) R preferably represents C₇ to C₂₀, preferably C₈ to C₁₄ hydrocarbyl group,

R₀, R₁, R₂, R₃, R₄, R₅, and R₆, each, independently, represent hydrogen or C₁ to C₄ alkyl group.

R′₁ and R′₂, each, independently, represent C₁ to C₄ alkyl group.

The hydrocarbyl group for R may be a straight or branched chain alkyl, alkenyl or alkynyl group, especially alkyl or alkenyl.

Preferably, R represents a C₇ to C₁₂ aliphatic group; especially C₇ to C₁₀ aliphatic group. Examples of suitable alkyl groups include, for example, n-heptyl, n-octyl, n-nonyl, n-decyl, n-undecyl, n-dodecyl, 2-methyl-octyl, 4-ethyl-decyl, 8-methyl-decyl, and the like. The straight chain alkyl groups, such as n-heptyl, n-octyl, n-nonyl and n-decyl, are especially preferred. Examples of alkenyl groups include, for example, 2-hexenyl, 2-heptenyl, 2-octenyl, 2-nonenyl, 2′,6′-dimethyl-2′,6′-heptadienyl, 2′6′-dimethyl-2′-heptaenyl, and the like. The R group may also be substituted by, for example, halo, hydroxy, carboxy, carboxamide and carboalkoxy.

The C₁ to C₄ alkyl group may be, for example, methyl, ethyl, n-propyl, isopropyl, n-butyl, tert-butyl, and the like. The preferred alkyl groups for R₀, and for R₁ to R₆ and for R′₁ and R′₂ are alkyl having 1 or 2 carbon atoms, most especially ethyl. R₀, and R₁ to R₆ may also, preferably, all be hydrogen.

Specific enhancer compounds (i) include, for example, 2-n-heptyl-1,3-dioxolane, 2-n-nonyl-1,3-dioxolane, 2-n-undecyl-1,3-dioxolane, 2-n-nonyl-1,3-dioxane, 2-n-undecyl-1,3-dioxane, 2-n-heptylaldehyde-acetal, 2-n-octyl-aldehyde-acetal, 2-n-nonylaldehyde-acetal, 2-n-decylaldehyde-acetal, 3,7-dimethyl-2,6-octadienal (citral), citronal and the like. 2-n-nonyl-1,3-dioxolane (2-NND), available from the MacroChem Corp. under the trade name SEPA®-0009, and decanal dimethyl acetal (DDMA), are especially preferred.

The SPE (ii) are cyclic ketones and cyclic lactones and derivatives thereof, as disclosed in, for example, U.S. Pat. Nos. 5,023,252 and 5,731,303, the disclosures of which, are incorporated herein, in their entireties, by reference thereto.

The SPE compounds (ii) may be represented by the following formula (III): 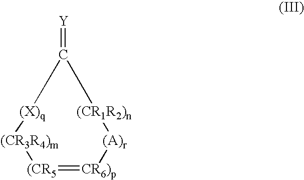
wherein X and Y are oxygen, sulfur or an imino group of the structure 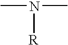
or=N-R, with the proviso that when Y is the imino group, X is an imino group, and when
Y is sulfur, X is sulfur or an imino group, A is group having the structure 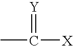
wherein X and Y are defined above,
m and n are integers having a value from 1 to 20 and the sum of m+n is not greater than 25,
p is an integer having a value of 0 or 1,
q is an integer having a value of 0 or 1,
r is an integer having a value of 0 or 1,
R represents hydrogen or a straight or branched chain alkyl group having from 1 to 6 carbon atoms, and,
R₁, R₂, R₃, R₄, R₅ and R₆, each, independently, represent hydrogen or a straight or branched chain alkyl group having from 1 to 6 carbon atoms, with the proviso that only one of R₁ to R₆ may be said alkyl group, and with the further provisos that,
when p, q and r have a value of 0 and Y is oxygen, m+n is at least 11,
when X is an imino group, q equals 1, Y is oxygen, and p and r are 0, then m+n is at least 11.

Examples of the alkyl group for R and R₁ to R₆ include methyl, ethyl, n-propyl, isopropyl, n-butyl, isobutyl, sec-butyl, amyl, hexyl, and the like.

Preferably, each of R and R₁ to R₆ are hydrogen atoms and X and Y each represent oxygen. These preferred compounds of formula (III) are, therefore, cyclic ketones (when q and r are each 0) or cyclic lactones.

Another preferred class of compounds of formula (III) may be represented by the following general formula (III-A): 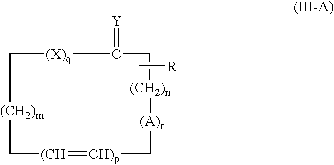
wherein X, Y, R, A, m, n, p, q and r, are as defined above.

Preferably, in formula (III-A), X and Y are each oxygen and R is preferably hydrogen.

Pentadecalactone is especially preferred as the SPE of type (ii).

The penetration enhancers of type (iii) include N-alkyl lactams, such as those disclosed in, for example, U.S. Pat. Nos. 4,316,893 and 4,424,210, the disclosures of which are incorporated herein, in their entirety, by reference thereto; and N-alkylazacycloheptanes, such as those disclosed in, for example, U.S. Pat. No. 5,204,339, the disclosure of which is incorporated herein, in its entirety, by reference thereto.

The N-alkyl lactams include, for example, compounds of the following formula (IV): 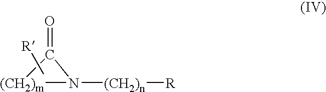
where R′ is H or a C₁ to C₄ alkyl group, R is C₁ to C₂ alkyl, phenyl or substituted phenyl, or the group 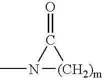
m is an integer of 3 to 7, n is 0 or an integer of 1 to 17, except that when m is 3, n is from 7 to 17, and R is preferably methyl.

A preferred class of lactams are represented by the following formula (IV-1): 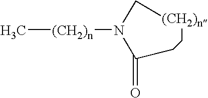
where n=0 or 1, and n″=0, 1 or 2.

Typical examples of compounds of formula (IV) include:

1-n-hexylazacyclopentan-2-one

1-n-heptaylazacyclopentan-2-one

1-n-octylazacyclopentan-2-one

1-n-nonylazacyclopentan-2-one

1-decylazacyclopentan-2-one

1-n-dodecylazacyclopentan-2-one

1-methylazacycloheptan-2-one

1-n-propylazacycloheptan-2-one

1-n-butylazacycloheptan-2-one

1-n-octylazacycloheptan-2-one

1-phenylazacyclopentan-2-one

1-(2-chlorophenyl)azacyclopentan-2-one

1,3-bis-(1-azacyclopentan-2-onyl)propane.

Of these, most preferred is 1-n-dodecyl-azacycloheptan-2-one, which is commercially available under the trade name, AZONE®

The N-alkylazacycloheptanes may be represented by the following formula (V): 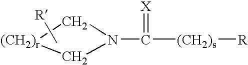
where X represents O or S, preferably O, R′ represents H or C₁ to C₄ alkyl; r is an integer of from 2 to 6, and s is 0 or an integer of 1 to 17.

Representative compounds of formula (V) include:

1-n-undecylformylazacycloheptane

1-n-decylformylazacycloheptane

1-n-octylformylazacycloheptane

1-n-nonylformylazacycloheptane

1-n-dodecylformylazacycloheptane

1-n-tetradecylformylazacycloheptane

1-n-hexadecylformylazacycloheptane

1-n-pentadecylformylazacycloheptane

1-n-heptadecylformylazacycloheptane

1-(16-methylhexadecyl) formylazacycloheptane.

Representative of the ester compounds (iv), include, for example, isopropyl myristate, isobutyl palmitate, 2-ethylhexyl ester of 4-(dimethylamino)benzoic acid (Padimate O), and the like.

The amount of the enhancer compound is selected to provide the desired delivery rate for the active compound but, taking into consideration such additional factors as, the amount of free ibuprofen, emulsion stability, and the like. Generally, amounts in the range of from about 1 to 20%, preferably from about 2 or 3 to about 12 or 15 percent, especially from about 5 to 12 or 15 percent, of the composition, will provide optimal flux rate and 24 hour payload of the active ingredient, and a homogeneous, oil-in-water emulsion.

In some cases, it is preferred to include a secondary surfactant to further promote the stability of the emulsion and cream formulation. Many of the known water-in-oil (W/O) type emulsifiers may be used for this purpose. Such W/O emulsifiers have a relatively low HLB value, such as from about 1 to about 8, preferably, from about 1.5 to about 7, more preferably, from about 2.5 to about 6, such as from about 2.5 to about 5, and therefore, would not themselves be expected to form the O/W emulsions of this invention. Since the ibuprofen salt functions as the primary emulsifier and forms by itself a homogeneous emulsion between the oil phase and the aqueous phase, the amount of the secondary emulsifier, when present, will be relatively low. One skilled in the art will be able to select a suitable amount of secondary emulsifying agent depending on such factors as the amounts of ibuprofen and primary emulsifier, the amount and type of oily phase and other additives in the composition. Generally, however, amounts within the range of from about 0.01 to 5%, preferably, from about 0.05 to about 4%, more preferably, from about 0.1 to about 2.5%, of secondary low HLB W/O emulsifier may be used in the emulsions and creams of the present invention.

The abbreviation “HLB” stands for hydrophilic lipophilic balance. The HLB system is well known in the art and is described in detail in “The HLB System, A Time-Saving Guide to Emulsifier Selection”, ICI Americas Inc., August 1984, which is incorporated herein by reference.

Exemplary secondary W/O emulsifiers for use in the present invention may be any cosmetically and pharmacologically acceptable oil soluble non-ionic or anionic (and in rare instances quaternary or amphoteric) surfactant which has a hydrophilic group (“tail”) at one end of the molecule. The preferred secondary emulsifiers are non-ionic.

Examples of suitable secondary emulsifiers include, for example, lipophilic non-ionic surfactant, such as, sorbitan fatty acid esters including certain sorbitan esters, preferably the sorbitan esters of C₁₆-C₂₂ saturated, unsaturated or branched chain fatty acids (usually comprised of mixtures of mono-, di-, tri-, etc. esters), such as, sorbitan monooleate (e.g., SPAN® 80), sorbitan sesquioleate (e.g., Arlacel® 83), sorbitan monoisostearate (e.g., CRILL® 6 made by Croda), sorbitan stearates (e.g., SPAN® 60), sorbitan triooleate (e.g., SPAN® 85), sorbitan tristearate (e.g., SPAN® 65), sorbitan dipalmitates (e.g., SPAN® 40), diglycerolsorbitan penta-2-ethylhexylate and diglycerolsorbitan tetra-2-ethylhexylate, etc.; glycerine or polyglycerine fatty acid esters including, for example, glyceryl monoesters, preferably glyceryl monoesters of C₁₆-C₂₂ saturated, unsaturated or branched chain fatty acids such as glyceryl monostearate, glyceryl monopalmitate, and glyceryl monobehenate; mono-cottonseed-fatty acid glyceryl ester, glyceryl monoerucate, glyceryl sesquioleate, glyceryl-alpha, alpha-oleate pyroglutamate and glyceryl monostearate monomalate; propylene glycol fatty acid esters including propylene glycol monostearate, as well as hydrogenated castor oil derivatives, and glycerol alkyl ether; hydrophilic non-ionic surfactant, such as, poly(ethylene oxide) (POE) sorbitan fatty acid esters including POE-sorbitan monooleate, POE-sorbitan monostearate, POE-sorbitan monooleate and POE-sorbitan tetraoleate, POE-sorbitol fatty acid esters including POE-sorbitol monolaurate, POE-sorbitol monooleate, POE-sorbitol pentaoleate and POE-sorbitol monostearate, etc., POE-glycerol fatty acid esters including POE-glyceryl monostearate, POE-glyceryl monoisostearate and POE-glyceryl triisostearate, POE fatty acid esters including POE monooleate, POE distearate, POE monodioleate and ethylene glycol distearate; POE alkyl ethers including POE lauryl ether, POE oleyl ether, POE stearyl ether, POE behenyl ether, POE 2-octyldodecyl ether and POE cholestanol ether, POE alkylphenyl ethers including POE octylphenyl ether etc., POE nonylphenylether and POE dinonylphenyl ether, pluaronics including pluronic, poly(ethylene oxide-polypropylene oxide) (POE-POP) alkyl ethers including POE-POP cetyl ether. POE-POP 2-decyltetradecyl ether, POE-POP monobutyl ether, POE-POP lanolin hydrate and POE-POP glycerol ether, tetra POE-tetra POP ethylenediamine condensates including tetronic, etc., POE castor oil hydrogenated castor oil derivatives including POE castor oil, POE hydrogenated castor oil, POE hydrogenated castor oil monoisostearate, POE hydrogenated castor oil triisostearate, POE hydrogenated castor oil monopyroglutamate monoisostearate diester, POE hydrogenated castor oil maleate, etc., POE beeswax/lanolin derivatives including POE sorbitol beeswax, etc., alkanol amides including coconut fatty acid diethanol amide, lauric acid monoethanol amide and fatty acid isopropanol amide; as well as POE propylene glycol fatty acid ester, POE alkyl amine, POE fatty acid amide, sucrose fatty acid ester, POE nonylphenylformaldehyde condensate, alkylethoxydimethylamine oxide and trioleyl phosphate.

Typical of these low HLB nonionic surfactants are alkoxylated, e.g., ethoxylated or propoxylated, fatty alcohols. In general, these alcohol derivatives contain a straight or branched chain alkyl group in the C₈₋₂₂, preferably C₁₀₋₂₀, more preferably C₁₂₋₂₀, range, and generally contain from about 1 to about 5 ethylene oxide (EO) groups per molecule.

Nonlimiting examples of such low HLB ethoxylated alcohol nonionic surfactants include stearic acid ethoxylated with 1 mole of ethylene oxide (i.e. steareth-1), steareth-2, steareth-3, steareth-4, steareth-5, ceteth-1, cetheth-2, ceteth-3, ceteth-4, ceteth-5, laureth-1, laureth-2, laureth-3, laureth-4, laureth-5, oleic acid ethoxylated with 1 mole or ethylene oxide (i.e. oleth-1), oleth-2, oleth-3, oleth-4, oleth-5, and mixtures thereof.

Other low HLB surfactants or emulsifiers which have been used in combination with the ibuprofen salt emulsifier include, for example, sorbitan stearate, glycerol monolaurate, Pluronic® L101, and Arlacel® P-135 (polyethylene glycol 1500 dihydroxystearate, available from ICI Americas, Inc). Glycerol monolaurate also functions as a preservative, therefore, use of this low HLB surfactant, provides the additional advantage of not requiring a separate preservative, as often include in pharmaceutical and cosmetic creams and ointments.

Pluronic is a poloxamer, a nonionic surfactant, and a block copolymer of propylene oxide and ethylene oxide. The propylene oxide block is sandwiched between two ethylene oxide blocks, as follows:
HO-(CH₂CH₂O)ₓ(CH₂CH₃CHO)_{y}(CH₂CH O)_{z}-H
where x,z=2-128,y=16-67. In Pluronic L101, x, z=7; y=54.

Other emulsifiers suitable for use in the present invention include silicone polymer emulsifiers such as alkyl dimethicone copolyols (e.g., Dow Corning Q2-5200); laurylmethicone copolyol; certain sucrose fatty acid esters, preferably sucrose esters of the C₁₆-C₂₂ saturated, unsaturated, and branched chain fatty acids such as sucrose trilaurate and sucrose distearate (e.g., Crodesta® F10), and certain polyglycerol esters of C₁₆-C₂₂ saturated, unsaturated or branched fatty acids such as diglycerol monooleate and tetraglycerol monooleate. Still other materials which may be useful as secondary emulsifier include ABA block copolymers of 12-hydroxystearic acid and polyethylene oxide, such as described in U.S. Pat. No. 4,875,927, issued to T. Tadros on Oct. 24, 1989, which is incorporated by reference herein. A representative material of this class useful as an emulsifier herein is available from Imperial Chemical Industries PLC as Arlacel® P135.

Detailed listings of low HLB surfactants can be found in McCutcheon's EMULSIFIERS AND DETERGENTS, North American Edition, 1984, McCutcheon Division, MC Publishing Company, incorporated herein by reference.

As indicated above, it has been found that inclusion of minor amounts of W/O emulsifiers promote the long range stability of the ibuprofen emulsions of the present invention. In this regard, stability is generally understood in the art as referring to the absence of phase separation, usually at elevated temperatures, e.g., about 40° C. or 50° C., and/or over extended periods of time, e.g., usually about 3 months, especially about 6 months or 1 year, or longer.

On the other hand, it is also generally known in the emulsification art, that stability of an emulsion is often a function of the method of preparation of the emulsion, for example, the degree of mixing and method of dispersing the ingredients of the emulsion and the particular processing equipment and preparative procedures. It is also understood that in some cases the prepared emulsions will be used rather quickly after preparation so that, even if phase separation might otherwise occur for a particular combination of ingredients after several months storage, or/and at temperatures in excess of about 30° C. or 40° C. or higher, addition of a stabilizing amount of secondary emulsifier may not be needed. Similarly, those skilled in the art may be able by, for example, changing the mode of mixing/dispersing the ingredients of the formulation and/or by using other processing equipment, enhance physical properties, such as product stability without the addition of secondary emulsifier.

In the present invention, therefore, it is understood that the method of mixing the ingredients of the emulsion and the processing equipment is not critical and any known or conventional method may be used, such as, for example, mechanical shaker, mechanical homogenization with or without heat (e.g., 60° C.), sonication, with or without heat, ultrasonication, and the like.

Generally, acceptable emulsions from the ibuprofen emulsified compositions of this invention, without addition of any other emulsifying (e.g., surface active) agents, can be formed using any mixing method and equipment for forming emulsions.

In order to convert the emulsion into a cream, it is usually necessary to add only a thickening agent to the emulsion, with suitable stirring or mixing. Examples of emulsifying thickening agents are well known to those skilled in the art. As representative of such thickening agents, mention may be made of, for example, the acrylic acid polymers, for example, the commercially available Carbopol® thickeners, e.g., Carbopol 974B, Carbopol 980, and the like, cellulosic ethers, such as, for example, hydroxypropyl cellulose, hydroxyethyl cellulose, and the like, guar gum, xanthan gum, and other polysaccharide thickeners, as well as inorganic thickeners/gelling agents. The amount of the thickening agent is not particularly critical and can be selected to provide the desired product consistency or viscosity to allow for easy application to the skin.

Generally, amounts of thickening agent up to about 5%, such as, for example, from 0.1 to about 3%, preferably, from about 0.2 to about 2%, of the composition will provide the desired effect.

Other additives, as needed, for functional or aesthetic attributes, may be included in the emulsions and creams of this invention so long as the objectives of the invention are not destroyed. As examples of such optional additives, mention may be made of, for example, perfumes and other odorants, preservatives (e.g., methyl paraben, ethyl paraben, DMDM hydantoin, glycerol monolaurate), colorants, and the like. When present, the optional additive(s) should preferably be used in the minimum amount to achieve the desired effect and without causing breaking or instability of the emulsion/cream composition. Typically, amounts less than about 3% of each additive, preferably up to about 2%, especially, up to about 1%. of additive may be included in the compositions of this invention.

It is also within the scope of the present invention to include one or more other active agents (e.g., substances providing pharmacological and/or therapeutic effects) including, for example, other NSAIDs, such as, heteroaryl acetic acids, such as, for example, tolmetin, diclofenac, ketorolac; arylpropionic acids, such as, for example, naproxen, flurbiprofen, ketoprofen, fenoprofen, oxaprozin; enolic acids, such as, for example, oxicams (e.g., piroxicam, tenoxicam), pyrazolidinediones (e.g., phenylbutazone, oxyphenthatrazone).

Other preferred classes of active agent which may be used in conjunction with ibuprofen include, for example, antiallergic, antihistarnine and decongestant compounds. Representative of the antiallergic compounds include, for instance, cromolyn, feniprane, lodoxamide, repirinast, tranilast, and the like, steroidal nasal antiallergic agents, such as, for example, beclomethasone, dexamthasone, flunisolide, triamcinolone acetonide, and the like. Examples of antihistamine compounds include, alkylamine derivatives, such as, acrivastine, brompheniramine, chlorpheniramine, tolpropamine, triprolidine and the like, aminoalkylethers, such as, clemastine, diphenhydramine, doxylamine, moxastine, and the like, ethylene diamine derivatives, such as, chloropyramine, chlorothen, histapyrrodine, pyrilamine, zolamine, and the like, piperazines, such as, chlorcyclizine, hydroxyzine, and the like, tricyclics, such as, fenethazine, isopromethazine, loratidine, and the like, azelastine, cetoxime, clemizole, ebastine, epinastine, fexofenadine, phenindamine, tritogualine, and the like. Representative examples of decongestants include, for example, amidephrine, cafaminol, ephedrine, epinephrine, fenoxazoline, oxymetazoline, phenyleprine HCI, pseudephedrine, tramazoline and the like. Further information on representative active agents can be found, for example, in the Merck Index, Twelfth Edition, 1996, published by Merck Research Laboratories Division of Merck & Co., Inc., the disclosure of which is incorporated herein, in its entirety, by reference thereto.

The following examples of ibuprofen emulsions and creams will provide assistance in understanding the invention. In these examples, thickener was added after all the other ingredients were emulsified using the procedure indicated. The resulting emulsions or creams were visually observed shortly after the completion of mixing to determine whether or not the composition has a yield value. A rating of “yes” indicates that the composition has a region of shear below which the composition behaves as “solid” or coherent mass; a rating of “no” indicates that the composition tends to flow, in the absence of applying shear; a rating of “marginal” indicates that only a very small shear region is required to cause the composition to become liquid-like. The emulsions and creams were also visually observed to determine whether or not the composition is homogeneous to the naked eye.

Some of the resulting compositions were also measured for the presence of crystals and were subjected to freeze-thaw cycles under the following conditions.

| | | 1 | 2 | 3 | 4 | 5 | 6 | 7 | 8 | 9 | 10 | 11 |
|---|---|---|---|---|---|---|---|---|---|---|---|---|
| | Ibuprofen | 5 | 5 | 5 | 5 | 5 | 5 | 5 | 5 | 5 | 5 | 5 |
| | Water | 81.2 | 81.2 | 81.2 | 81.2 | 81.2 | 82.5 | 82.0 | 81.2 | 81.2 | 81.2 | 81.2 |
| Bases | Triethanolamine | 1.8 | 1.8 | 1.8 | 1.8 | 1.8 | — | — | 1.8 | 1.8 | 1.8 | 1.8 |
| | Sodium hydroxide (25%) | — | — | — | — | — | 0.46 | — | — | — | — | — |
| | Diisopropanolamine | — | — | — | — | — | — | 1 | — | — | — | — |
| Thickeners | Carbopol ® 974P | 1 | 1 | 1 | 1 | 1 | 1 | 1 | 1 | 1 | 1 | 1 |
| | Carbopol 980 | — | — | — | — | — | — | — | — | — | — | — |
| | Hydroxyethylcellulose 250 M | — | — | — | — | — | — | — | — | — | — | — |
| | Hydroxyethylcellulose 250 HX | — | — | — | — | — | — | — | — | — | — | — |
| | Hydroxypropylcellulose (Klucel ® H) | — | — | — | — | — | — | — | — | — | — | — |
| Enhancers | SEPA ® 0009 | — | — | — | — | — | 10 | 10 | — | — | — | 10 |
| | SEPA DDMA | — | — | — | — | — | — | — | — | — | — | — |
| | Padimate ® O | 10 | — | — | — | — | — | — | — | — | — | — |
| | Isopropyl myristate | — | 10 | — | — | — | — | — | — | — | — | — |
| | Azone ® | — | — | 10 | — | — | — | — | — | — | — | — |
| | Pentadecalactone | — | — | — | 10 | — | — | — | — | — | — | — |
| | Dodecyl dimethylaminopropionate | — | — | — | — | 10 | — | — | — | — | — | — |
| Oils | Light mineral oil | — | — | — | — | — | — | — | 10 | — | — | — |
| | Miglyol ® 812N | — | — | — | — | — | — | — | — | 10 | — | — |
| | Cyclomethicone | — | — | — | — | — | — | — | — | — | 10 | — |
| Stabilizers | Sorbitan stearate | 1 | 1 | 1 | 1 | 1 | 1 | 1 | 1 | 1 | 1 | 1 |
| | Pluronic ® L101 | — | — | — | — | — | — | — | — | — | — | — |
| | Arlacel P-135 | — | — | — | — | — | — | — | — | — | — | — |
| | Glycerol monolaurate | — | — | — | — | — | — | — | — | — | — | — |
| Preservatives | Methyl paraben | — | — | — | — | — | — | — | — | — | — | — |
| | Propyl paraben | — | — | — | — | — | — | — | — | — | — | — |
| | DMDM Hydantoin | — | — | — | — | — | — | — | — | — | — | — |
| | Fragrance | — | — | — | — | — | — | — | — | — | — | — |
| | Total | 100 | 100 | 100 | 100 | 100 | 100 | 100 | 100 | 100 | 100 | 100 |
| | | 12 | 13 | 14 | 15 | 16 | 17 | 18 | 19 | 20 | 21 | 22 |
| | Ibuprofen | 5 | 5 | 5 | 5 | 5 | 5 | 5 | 5 | 5 | 5 | 5 |
| | Water | 81.2 | 81.0 | 81.0 | 81.2 | 81.2 | 86.2 | 81.19 | 79 | 84.2 | 79 | 84.2 |
| Bases | Triethanolamine | 1.8 | 1.8 | 1.8 | 1.8 | 1.8 | 1.8 | 1.81 | 3.6 | 3.6 | 3.6 | 3.6 |
| | Sodium hydroxide (25%) | — | — | — | — | — | — | — | — | — | — | — |
| | Diisopropanolamine | — | — | — | — | — | — | — | — | — | — | — |
| Thickeners | Carbopol ® 974P | 1 | 1 | 1 | 1 | 1 | 1 | 1 | — | — | — | — |
| | Carbopol 980 | — | — | — | — | — | — | — | 1 | 1 | 1 | 1 |
| | Hydroxyethylcellulose 250 M | — | — | — | — | — | — | — | — | — | — | — |
| | Hydroxyethylcellulose 250 HX | — | — | — | — | — | — | — | — | — | — | — |
| | Hydroxypropylcellulose (Klucel ® H) | — | — | — | — | — | — | — | — | — | — | — |
| Enhancers | SEPA ® 0009 | | 10 | 10 | 10 | 10 | — | — | — | — | — | — |
| | SEPA DDMA | 10 | — | — | — | — | 5 | 10 | 10 | 5 | 10 | 5 |
| | Padimate ® O | — | — | — | — | — | — | — | — | — | — | — |
| | Isopropyl myristate | — | — | — | — | — | — | — | — | — | — | — |
| | Azone ® | — | — | — | — | — | — | — | — | — | — | — |
| | Pentadecalactone | — | — | — | — | — | — | — | — | — | — | — |
| | Dodecyl dimethylaminopropionate | — | — | — | — | — | — | — | — | — | — | — |
| Oils | Light mineral oil | — | — | — | — | — | — | — | — | — | — | — |
| | Miglyol ® 812N | — | — | — | — | — | — | — | — | — | — | — |
| | Cyclomethicone | — | — | — | — | — | — | — | — | — | — | — |
| Stabilizers | Sorbitan stearate | 1 | 1 | 1 | — | 1 | 1 | 1 | 1 | 1 | 1 | 1 |
| | Pluronic ® L101 | — | — | — | — | — | — | — | — | — | — | — |
| | Arlacel P-135 | — | — | — | — | — | — | — | — | — | — | — |
| | Glycerol monolaurate | — | — | — | 1 | — | — | — | — | — | — | — |
| Preservatives | Methyl paraben | — | 0.2 | — | — | — | — | — | — | — | — | — |
| | Propyl paraben | — | — | 0.2 | — | — | — | — | — | — | — | — |
| | DMDM Hydantoin | — | — | — | — | — | — | — | — | — | — | — |
| | Fragrance | — | — | — | — | — | — | — | 0.4 | 0.2 | 0.4 | 0.2 |
| | Total | 100 | 100 | 100 | 100 | 100 | 100 | 100 | 100 | 100 | 100 | 100 |
| | | 23 | 24 | 25 | 26 | 27 | 28 | 29 | 30 | 31 | 32 | 33 |
| | Ibuprofen | 5 | 5 | 5 | 5 | 5 | 5 | 5 | 5 | 5 | 5 | 5 |
| | Water | 78.2 | 78.2 | 79.2 | 78.2 | 78.2 | 78.2 | 79.7 | 80.8 | 80.4 | 83.2 | 82.2 |
| Bases | Triethanolamine | 3.6 | 3.6 | 3.6 | 3.6 | 3.6 | 3.6 | 3.6 | 1.8 | 1.8 | 1.8 | 1.8 |
| | Sodium hydroxide (25%) | — | — | — | — | — | — | — | — | — | — | — |
| | Diisopropanolamine | — | — | — | — | — | — | — | — | — | — | — |
| Thickeners | Carbopol ® 974P | — | — | — | 1 | 1 | 1 | 1 | 1 | 1 | — | — |
| | Carbopol 980 | 1 | 1 | 1 | — | — | — | — | — | — | — | — |
| | Hydroxyethylcellulose 250 M | — | — | — | — | — | — | — | — | — | — | — |
| | Hydroxyethylcellulose 250 HX | — | — | — | — | — | — | — | — | — | — | — |
| | Hydroxypropylcellulose (Klucel ® H) | — | — | — | — | — | — | — | — | — | — | 1 |
| Enhancers | SEPA ® 0009 | — | — | — | — | — | — | — | 10 | 10 | 10 | 10 |
| | SEPA DDMA | 10 | 10 | 10 | 10 | 10 | 10 | 10 | — | — | — | — |
| | Padimate ® O | — | — | — | — | — | — | — | — | — | — | — |
| | Isopropyl myristate | — | — | — | — | — | — | — | — | — | — | — |
| | Azone ® | — | — | — | — | — | — | — | — | — | — | — |
| | Pentadecalactone | — | — | — | — | — | — | — | — | — | — | — |
| | Dodecyl dimethylaminopropionate | — | — | — | — | — | — | — | — | — | — | — |
| Oils | Light mineral oil | — | — | — | — | — | — | — | — | — | — | — |
| | Miglyol ® 812N | — | — | — | — | — | — | — | — | — | — | — |
| | Cyclomethicone | — | — | — | — | — | — | — | — | — | — | — |
| Stabilizers | Sorbitan stearate | 2 | — | — | 2 | 2 | 2 | 0.5 | 1.0 | 1.0 | — | — |
| | Pluronic ® L101 | — | 2 | 1 | — | — | — | — | — | — | — | — |
| | Arlacel P-135 | — | — | — | — | — | — | — | — | — | — | — |
| | Glycerol monolaurate | — | — | — | — | — | — | — | — | — | — | — |
| Preservatives | Methyl paraben | — | — | — | — | — | — | — | 0.2 | — | — | — |
| | Propyl paraben | — | — | — | — | — | — | — | 0.2 | — | — | — |
| | DMDM Hydantoin | — | — | — | — | — | — | — | — | 0.8 | — | — |
| | Fragrance | 0.2 | 0.2 | 0.2 | 0.2 | 0.2 | 0.2 | 0.2 | — | — | — | — |
| | Total | 100 | 100 | 100 | 100 | 100 | 100 | 100 | 100 | 100 | 100 | 100 |
| | | 34 | 35 | 36 | 37 | 38 | 39 | 40 | 41 | 42 | 43 | 44 |
| | Ibuprofen | 5 | 5 | 5 | 5 | 4.8 | 1 | 1 | 1 | 5 | 5 | 5 |
| | Water | 83.2 | 83.5 | 82.2 | 81.2 | 76.4 | 87.4 | 87.4 | 87.4 | 79.4 | 79.4 | 79.4 |
| Bases | Triethanolamine | 1.8 | — | 1.8 | 1.8 | 6.9 | 0.36 | 0.36 | 0.36 | 3.6 | 3.6 | 3.6 |
| | Sodium hydroxide (25%) | — | 1.5 | — | — | — | — | — | — | — | — | — |
| | Diisopropanolamine | — | — | — | — | — | — | — | — | — | — | — |
| Thickeners | Carbopol ® 974P | — | — | 1 | 1 | 1.3 | 1 | 1 | 1 | 1 | 1 | 1 |
| | Carbopol 980 | — | — | — | — | — | — | — | — | — | — | — |
| | Hydroxyethylcellulose 250 M | — | — | — | — | — | — | — | — | — | — | — |
| | Hydroxyethylcellulose 250 HX | — | — | — | — | — | — | — | — | — | — | — |
| | Hydroxypropylcellulose (Klucel ® H) | — | — | — | — | — | — | — | — | — | — | — |
| Enhancers | SEPA ® 0009 | 10 | 10 | 10 | 10 | 9.6 | 10 | 10 | 10 | — | — | — |
| | SEPA DDMA | — | — | — | — | — | — | — | — | — | — | — |
| | Padimate ® O | — | — | — | — | — | — | — | — | — | — | — |
| | Isopropyl myristate | — | — | — | — | — | — | — | — | — | — | — |
| | Azone ® | — | — | — | — | — | — | — | — | — | — | — |
| | Pentadecalactone | — | — | — | — | — | — | — | — | — | — | — |
| | Dodecyl dimethylaminopropionate | — | — | — | — | — | — | — | — | — | — | — |
| Oils | Light mineral oil | — | — | — | — | — | — | — | — | 10 | — | — |
| | Miglyol ® 812N | — | — | — | — | — | — | — | — | — | 10 | — |
| | Cyclomethicone | — | — | — | — | — | — | — | — | — | — | 10 |
| Stabilizers | Sorbitan stearate | — | — | — | 1 | 1 | 0.2 | 0.2 | 0.2 | 1 | 1 | 1 |
| | Pluronic ® L101 | — | — | — | — | — | — | — | — | — | — | — |
| | Arlacel P-135 | — | — | — | — | — | — | — | — | — | — | — |
| | Glycerol monolaurate | — | — | — | — | — | — | — | — | — | — | — |
| Preservatives | Methyl paraben | — | — | — | — | — | — | — | — | — | — | — |
| | Propyl paraben | — | — | — | — | — | — | — | — | — | — | — |
| | DMDM Hydantoin | — | — | — | — | — | — | — | — | — | — | — |
| | Fragrance | — | — | — | — | — | — | — | — | — | — | — |
| | Total | 100 | 100 | 100 | 100 | 100 | 100 | 100 | 100 | 100 | 100 | 100 |
| | | 45 | 46 | 47 | 48 | 49 | 50 | 51 | 52 | 53 | 54 | 55 |
| | Ibuprofen | 5 | 5 | 1 | 2.5 | 5 | 7.5 | 10 | 5 | 5 | 1 | 1 |
| | Water | 79.4 | 79.4 | 86.6 | 84.6 | 80.2 | 77.8 | 74.4 | 82.2 | 81.2 | 86.1 | 85.8 |
| Bases | Triethanolamine | 3.6 | 3.6 | 1.16 | 1.4 | 1.8 | 2.2 | 2.6 | 1.8 | 1.8 | 1.75 | 2.01 |
| | Sodium hydroxide (25%) | — | — | — | — | — | — | — | — | — | — | — |
| | Diisopropanolamine | — | — | — | — | — | — | — | — | — | — | — |
| Thickeners | Carbopol ® 974P | 1 | 1 | 1 | 1 | 1 | 1 | 1 | — | 1 | 1 | 1 |
| | Carbopol 980 | — | — | — | — | — | — | — | — | — | — | — |
| | Hydroxyethylcellulose 250 M | — | — | — | — | — | — | — | — | — | — | — |
| | Hydroxyethylcellulose 250 HX | — | — | — | — | — | — | — | — | — | — | — |
| | Hydroxypropylcellulose (Klucel ® H) | — | — | — | — | — | — | — | — | — | — | — |
| Enhancers | SEPA ® 0009 | 10 | — | 10 | 10 | 10 | 10 | 10 | 10 | 10 | 10 | 10 |
| | SEPA DDMA | — | 10 | — | — | — | — | — | — | — | — | — |
| | Padimate ® O | — | — | — | — | — | — | — | — | — | — | — |
| | Isopropyl myristate | — | — | — | — | — | — | — | — | — | — | — |
| | Azone ® | — | — | — | — | — | — | — | — | — | — | — |
| | Pentadecalactone | — | — | — | — | — | — | — | — | — | — | — |
| | Dodecyl dimethylaminopropionate | — | — | — | — | — | — | — | — | — | — | — |
| Oils | Light mineral oil | — | — | — | — | — | — | — | — | — | — | — |
| | Miglyol ® 812N | — | — | — | — | — | — | — | — | — | — | — |
| | Cyclomethicone | — | — | — | — | — | — | — | — | — | — | — |
| Stabilizers | Sorbitan stearate | 1 | 1 | 0.2 | 0.5 | 1 | 1.5 | 2 | — | — | 0.2 | 0.2 |
| | Pluronic ® L101 | — | — | — | — | — | — | — | — | — | — | — |
| | Arlacel P-135 | — | — | — | — | — | — | — | 1 | 1 | — | — |
| | Glycerol monolaurate | — | — | — | — | — | — | — | — | — | — | — |
| Preservatives | Methyl paraben | — | — | — | — | — | — | — | — | — | — | — |
| | Propyl paraben | — | — | — | — | — | — | — | — | — | — | — |
| | DMDM Hydantoin | — | — | — | — | — | — | — | — | — | — | — |
| | Fragrance | — | — | — | — | 1 | — | — | — | — | — | — |
| | Total | 100 | 100 | 100 | 100 | 100 | 100 | 100 | 100 | 100 | 100 | 100 |

| | | 56 | 57 | 58 | 59 | 60 | 61 | 62 | 63 | 64 | 65 | 66 | 67 |
|---|---|---|---|---|---|---|---|---|---|---|---|---|---|
| | Ibuprofen | 2.5 | 2.5 | 5 | 5 | 7.5 | 5 | 5 | 5 | 7.5 | 10 | 7.5 | 7.5 |
| | Water | 83.6 | 83.2 | 79.4 | 78.8 | 75.2 | 81.2 | 81.2 | 81.2 | 77.6 | 74.1 | 76.0 | 77.6 |
| Bases | Triethanolamine | 2.45 | 2.83 | 3.6 | 4.2 | 4.76 | 1.8 | 1.8 | 1.8 | 2.36 | 2.94 | 2.99 | 2.36 |
| | Sodium hydroxide (25%) | — | — | — | — | — | — | — | — | — | — | — | — |
| | Diisopropanolamine | — | — | — | — | — | — | — | — | — | — | — | — |
| Thick- | Carbopol ® 974P | 1 | 1 | 1 | 1 | 1 | 1 | — | — | 1 | 1 | 2 | — |
| eners | Carbopol 980 | — | — | — | — | — | — | — | — | — | — | — | 1 |
| | Hydroxyethylcellulose 250 M | — | — | — | — | — | — | 2 | — | — | — | — | — |
| | Hydroxyethylcellulose 250 HX | — | — | — | — | — | — | — | 1 | — | — | — | — |
| | Hydroxypropylcellulose (Klucel ® | — | — | — | — | — | — | | | — | — | — | — |
| | H) | | | | | | | | | | | | |
| Enhancers | SEPA ® 0009 | 10 | 10 | 10 | 10 | 10 | 10 | 10 | 10 | 10 | 10 | 10 | 10 |
| | SEPA DDMA | — | — | — | — | — | — | — | — | — | — | — | — |
| | Padimate ® O | — | — | — | — | — | — | — | — | — | — | — | — |
| | Isopropyl myristate | — | — | — | — | — | — | — | — | — | — | — | — |
| | Azone ® | — | — | — | — | — | — | — | — | — | — | — | — |
| | Pentadecalactone | — | — | — | — | — | — | — | — | — | — | — | — |
| | Dodecyl dimethylaminopropionate | — | — | — | — | — | — | — | — | — | — | — | — |
| Oils | Light mineral oil | — | — | — | — | — | — | — | — | — | — | — | — |
| | Miglyol ® 812N | — | — | — | — | — | — | — | — | — | — | — | — |
| | Cyclomethicone | — | — | — | — | — | — | — | — | — | — | — | — |
| Stabilizers | Sorbitan stearate | 0.5 | 0.5 | 1 | 1 | 1.5 | 1 | 1 | 1 | 1.5 | 2 | 1.5 | 1.5 |
| | Pluronic ® L101 | — | — | — | — | — | — | — | — | — | — | — | — |
| | Arlacel P-135 | — | — | — | — | — | — | — | — | — | — | — | — |
| | Glycerol monolaurate | — | — | — | — | — | — | — | — | — | — | — | — |
| Preser- | Methyl paraben | — | — | — | — | — | — | — | — | — | — | — | — |
| vatives | Propyl paraben | — | — | — | — | — | — | — | — | — | — | — | — |
| | DMDM Hydantoin | — | — | — | — | — | — | — | — | — | — | — | — |
| | Fragrance | — | — | — | — | — | — | — | — | — | — | — | — |
| | Total | 100 | 100 | 100 | 100 | 100 | 100 | 100 | 100 | 100 | 100 | 100 | 100 |

The composition of Example 11 (ibuprofen, 5%, 2-n-nonyl-1,3-dioxolane, 10%, sorbitan monooleate, 1%, Carbopol 974P, 1%, triethanolamine, 1.8%, water, QS 100) was subjected to an in vitro test (standard Franz cell), to measure delivery of ibuprofen. For comparison, two ibuprofen containing gel compositions were similarly tested:

| | Gel A | Gel B |
|---|---|---|
| Ibuprofen | 5% | 5% |
| 2-n-nonyl-1,3-dioxolane | 0 | 10 |
| Hydroxypropylcellulose | 2 | 2 |
| NaOH | QS pH = 7 | QS pH = 7 |
| Solvent | | |
| Ethanol/Propylene Glycol/Water | QS 100 | QS 100 |
| (70:20:10) | | |

Furthermore, to test for the stability of the cream formulations of this invention, a composition, identical to that of Example 11, but prepared approximately 22 months earlier, was also tested. The results (ibuprofen permeation, μg/cm² vs. time, hours) are shown in the attached Fig.

From the Figure, it is seen that the freshly prepared and 22 month old cream formulations according to this invention provide comparable delivery to the enhancer-containing gel (Gel A). The long term stability of the invention cream formulations is an important property for a commercially feasible product.

1. An ibuprofen O/W emulsion composition comprising:
an emulsifying effective amount of ibuprofen salt as substantially the only O/W emulsifier;
oily substance; and water.

2. The composition of claim 1, wherein said oily phase comprises a skin penetration enhancer effective for enhancing the permeation of ibuprofen through mammalian skin.

3. The composition of claim 2, wherein said skin penetration enhancer is at least one compound selected from the group consisting of:
(i) C₇ to C₁₄-hydrocarbyl substituted 1,3-dioxolane, 1,3-dioxane or acetal;
(ii) macrocyclic ketones and lactones and derivatives thereof;
(iii) N-alkyl lactams and N-alkyl azacycloheptanes;
(iv) fatty acid esters.

4. The composition of claim 1, wherein said base comprises at least one organic base.

5. The composition of claim 4, wherein the organic base comprises an amine compound.

6. The composition of claim 1, wherein said base comprises at least one inorganic base.

7. The composition of claim 6, wherein said inorganic base is an alkali metal compound.

8. The composition of claim 1, further comprising a minor amount of a W/O emulsifier.

9. The composition of claim 8, wherein the W/O emulsifier is at least one nonionic surfactant compound selected from the group consisting of sorbitan fatty acid esters, glycerine fatty acid esters, polyglycerine fatty acid esters, propylene glycol fatty acid esters, alkanolamides and alkoxylated alcohols.

10. The composition of claim 8, wherein said W/O emulsifier is at least one compound selected from the group consisting of sorbitan stearate, glycerol monolaurate, ethylene oxide/propylene oxide block copolymer, and polyethylene glycol dihydroxystearate.

11. A cream formulation effective for the topical administration of ibuprofen, comprising,
an emulsifying and therapeutically effective amount of ibuprofen,
a base effective for forming an ibuprofen salt,
an oily substance,
thickener and water,
wherein the ibuprofen salt is present in an amount effective to form an oil-in-water emulsion without assistance of other emulsifying agent.

12. The ibuprofen cream according to claim 11, which comprises from about 1 to about 10% by weight of ibuprofen.

13. The ibuprofen cream according to claim 12, wherein said oily substance comprises at least one skin penetration enhancing compound effective for promoting transdermal delivery of ibuprofen.

14. The ibuprofen cream according to claim 13, wherein said at least one skin penetration enhancing compound is selected from the group consisting of:
(i) C₇ to C₁₄-hydrocarbyl substituted 1,3-dioxolane, 1,3-dioxane or acetal;
(ii) macrocyclic ketones and lactones and derivatives thereof;
(iii) N-alkyl lactams and N-alkyl azacycloheptanes;
(iv) fatty acid esters.

15. The ibuprofen cream according to claim 12, which farther comprises a minor amount of at least one W/O emulsifying surfactant.

16. The ibuprofen cream according to claim 15, wherein said W/O emulsifying surfactant is selected from the group consisting of sorbitan stearate, glycerol monolaurate, Pluronic® LI 101, and ArlacelO P-135 (polyethylene glycol 1500 dihydroxystearate.

17. The ibuprofen cream according to claim 11, which consists essentially of
from about 1 to about 10% of ibuprofen,
from about 2 to about 10% of said oily substance,
base in an amount to provide a pH in the range of from about 4 to about 8, and
thickening agent,
0 to about 5% of low HLB W/O emulsifier,
0 to about 3% of one or more additives selected from the group consisting of odorants, colorants and preservatives.

18. The ibuprofen cream according to claim 17, wherein said oily substance is at least one skin penetration enhancing compound effective for enhancing the transdermal administration of ibuprofen across mammalian skin.

19. The ibuprofen cream according to claim 18, wherein said skin penetration enhancing compound is at least one compound selected from the group consisting of
(i) C₇ to C₁₄-hydrocarbyl substituted 1,3-dioxolane, 1,3-dioxane or acetal;
(ii) macrocyclic ketones and lactones and derivatives thereof;
(iii) N-alkyl lactams and N-alkyl azacycloheptanes;
(iv) fatty acid esters.

20. An ibuprofen emulsion consisting essentially of
from about 1 to 10% by weight of ibuprofen,
from about 0.3 to about 5% by weight of a basic substance selected from the group consisting of sodium hydroxide, triethanolamine, and diisopropanolamine,
from about 5 to 10% by weight of oily skin penetration enhancing compound selected from the group consisting of 2-n-nonyl-1,3-dioxolane, decanaldimethyl acetal, isopropyl myristate, 1-dodecylazacycloheptan-2-one and pentadecalactone,
0 to about 3% by weight of secondary W/O emulsifying agent,
0 to 2% by weight of preservative,
0 to 1% by weight of odorant,
0 to 1% by weight of colorant, and
balance water.

21. An ibuprofen cream consisting essentially of
from about 1 to 10% by weight of ibuprofen,
from about 0.3 to about 5% by weight of a basic substance selected from the group consisting of sodium hydroxide, triethanolamine, and diisopropanolamine,
from about 5 to 10% by weight of oily skin penetration enhancing compound selected from the group consisting of 2-n-nonyl-1,3-dioxolane, decanaldimethyl acetal, isopropyl myristate, 1-dodecylazacycloheptan-2-one and pentadecalactone,
a cream forming effective amount of thickening agent,
0 to about 3% by weight of secondary W/O emulsifying agent,
0 to 2% by weight of preservative,
0 to 1% by weight of odorant,
0 to 1% by weight of colorant, and
balance water.

22. A method for preparing an ibuprofen containing emulsion without addition of O/W emulsifying agent, comprising
combining ibuprofen with water, oily substance and a base in an amount effective to partially neutralize the carboxylic group of ibuprofen, and
mixing the resulting combination until an O/W emulsion is formed.

23. A method for preparing an ibuprofen cream which does not contain any or only insubstantial amount of O/W emulsifying agent, which comprises,
combining ibuprofen with water, oily substance and a base in an amount effective to partially neutralize the carboxylic group of ibuprofen, and
mixing the resulting combination until an O/W emulsion is formed, and
mixing the resulting emulsion with a thickening agent.
